(19) United States
(12) Patent Application Publication
(10) Pub. No.: US 2010/0161078 A1
(43) Pub. Date: Jun. 24, 2010

(54) SIMPLIFIED PROSTHETIC DEVICE

(75) Inventor: Joy Danielle Clark, Des Moines, IA (US)

Correspondence Address: United States Army Medical Research;and Material Command MCMR-ZA-J, 504 Scott Street, Fort Detrick, MD, 21702-5012, US

(73) Assignee: United States Army, as represented by the Secretary of the Army, Fort Detrick, MD (US)

(21) Appl. No.: 12/640,300

(22) Filed: Dec. 17, 2009

Related U.S. Application Data
(60) Provisional application No. 61/139,577, filed on Dec. 20, 2008.

(51) Int. Cl. A61F 2/54

(52) U.S. Cl. 623/65

(57) ABSTRACT

A simplified prosthetic device and methods of use thereof is disclosed with splinting means; an elongate static arm fixedly attached to said splinting means; an elongate mobile arm with a first end and second end, said mobile arm being flexibly attached at said first end to said static arm; an elastic element for allowing re-extension of said mobile arm and attached to said mobile arm and an anchor point; means for translating gross body movements to the mobile arm; and an attachment being attached to the second end of the mobile arm.

### CROSS-REFERENCE TO RELATED APPLICATIONS

This application claims priority to U.S. Provisional Application 61/139,577, filed on Dec. 20, 2008, the entirety of which being incorporate herein by this reference.

### RIGHTS

This invention was made with support from the United States Government, specifically, the United States Army Institute of Surgical Research, and accordingly, the United States has certain rights in this invention.

### FIELD AND BACKGROUND OF THE INVENTION

The present invention relates generally to prosthetic and orthotic devices, and specifically to a simplified, harness-free, body-powered, upper-extremity prosthesis.

A body-powered prosthesis is powered and controlled by gross body movements. Patients are able to capture the movements of their shoulder, upper arm or chest by a harness system, which is attached to a cable that is connected to a terminal device (hook or hand). An elbow system can be added to provide the patient additional functionality. For a good over-view of progressive upper limb prosthetics see (Lake C, 2006)

Because of its simple design, this type of prosthesis is highly durable and easy to maintain. However, wearers typically complain that these systems are uncomfortable due to the restrictive harness. Therefore, a more comfortable body-powered prosthetic device is needed that retains a simple design.

Another disadvantage of known body-powered prostheses is that the particular disability of a patient may disqualify him or her from being able to use the device properly.

To be a candidate for known body-powered prostheses, a patient must possess at least one or more of the following gross body movements: glenohumeral flexion, scapular abduction or adduction, shoulder depression and elevation, and chest expansion. Additionally, a patient must have sufficient range of motion, musculature and residual limb strength to successfully manipulate the prosthesis. Therefore, there exists a need for a body-powered prosthesis that reduces the need for multiple gross body movements and range of motion to function properly.

Frequently, patients with severe burns or tissue trauma to their remaining limbs are not able to receive a prosthetic device until they are fully healed. This could take months, forestalling the physical therapy and training necessary for a patient to regain function and independence. Therefore, a need exists for a simple prosthesis that may be comfortably worn by a recently injured patient and which requires minimal gross body movement or range of motion to function properly.

The needs noted above are presently unmet by upper-extremity prosthesis known in the art.

U.S. Pat. No. 1,989,960 to Wheeler et al. shows a complicated, body-powered, prosthesis requiring a plurality of beveled, pivoting, co-acting joint sections and a shoulder harness for mounting the device on the user.

The prosthetic arm in U.S. Pat. No. 2,834,024 to Aveni requires a supporting harness, hollow arm sections, and a pulley.

U.S. Pat. No. 1,366,453 to Henning features a device that also requires a shoulder harness, and a saddle adapted to rest upon the user's shoulder.

Fishbein et al., in U.S. Pat. No. 2,537,338, disclose a prosthetic arm with a complex elbow assembly requiring rotatable cam members with engaging teeth and a harness adapted to be secured on the shoulders of the wearer.

The lateral thumb hand prosthesis disclosed in U.S. Pat. No. 4,258,411 to Bell requires a prosthetic hand and lateral thumb connected to a harness on the shoulder opposite the side of the amputation by way of the posterior elbow.

It is, therefore an object of this invention to provide a simple, comfortable, harness-free, body-powered upper extremity prosthesis capable of being worn and used by patients of limited physical ability.

### SUMMARY OF THE INVENTION

The device provides the patient with an artificial elbow that may be “bent” through flexion and/or abduction of the patient's natural upper arm. The device includes a base or support that is fixed to the upper arm. The base may comprise a pair of semi-cylindrical plastic shells with hook and loop straps for attachment to the upper arm. A hinged member includes a pair of elongate arms pivotally joined together. One elongate arm is securely fixed to the base and functions as the lower portion of the patient's upper arm. The pivot on the hinged member functions as the elbow. The other elongate arm functions as the forearm.

The end of the “forearm” member may be used to attach different articles that may be used by the patient, such as eating utensils, toothbrush, etc. Near the end of the “forearm” member a thread, wire or the like is attached. An eyelet is fixed to the base on the inner side, i.e., toward the chest area. The thread leads from its attachment point on the “forearm” member, through the eyelet on the base and then to a second attachment point near the patient's waist, on the same side as the device. The second attachment point may take the form of a loop for a belt to pass through, a piece of foam that may be tucked in a patient's waist line, or similar anchoring devices. When the patient flexes or abducts his/her upper arm, the “forearm” member pivots toward the patient, in the manner of an elbow. Thus, the patient is able to perform otherwise impossible tasks.

The various features of novelty that characterize the invention are pointed out with particularity in the claims annexed to and forming a part of this disclosure. For a better understanding of the invention, its operating advantages and specific objects attained by its uses, reference is made to the accompanying drawings and descriptive in which a preferred embodiment of the invention is illustrated.

### BRIEF DESCRIPTION OF THE DRAWINGS

In the drawings:

FIG. 1 shows an exploded, perspective view of an embodiment of the invention with a spoon attachment.

FIG. 2 shows an overhead, plan view of an embodiment of the invention with a spoon attachment.

FIG. 3 shows a drawing of an embodiment of the invention with a fork attachment in use by a patient

### DESCRIPTION OF THE PREFERRED EMBODIMENTS

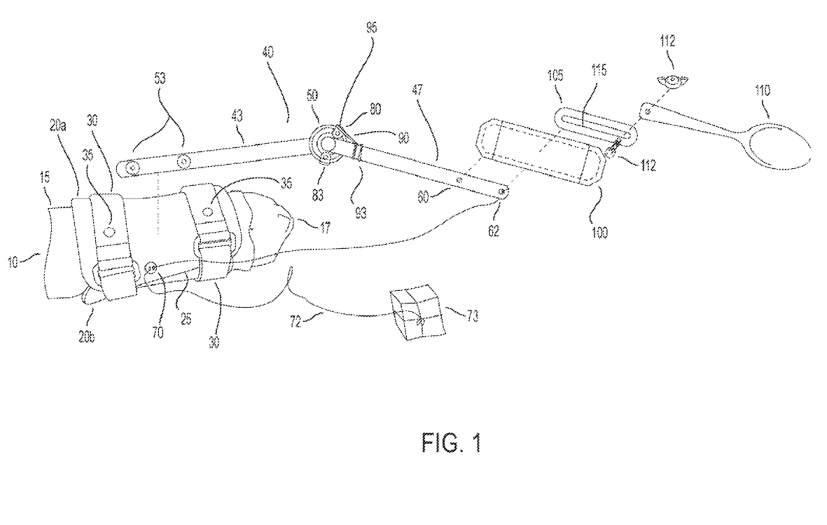

In FIG. 1 the simplified prosthetic device 10 as worn on the amputee's limb 15 is shown with its constituent elements in exploded view. The limb is covered by an optional compression sleeve 17. The device 10 is secured to the limb 15 by splinting means 20, preferably of 1% perforated thermoplastic moulding such as that sold by Patterson Medical Holdings, inc., 270 Remington Boulevard Suite C Bolingbrook Ill. 60440 (Sammons Preston Rolyan) under the trademark POLYFORM™ splinting material. In one embodiment of the invention, the splinting means 20 is composed of two, curved, over-lapping halves (20a and 20b), wherein the smaller (medial/anterior) splint half 20b is manufactured so as to fit within the peripheral edge 25 of the larger (lateral/posterior) splint half 20a in an arrangement known in the art as a “clam shell” splint. The larger splint half 20a should overlap the smaller splint half 20b preferably by ½″ to 1 inch.

Continuing with FIG. 1 of the preferred embodiment of the invention, we see that the smaller splint half 20b and the larger splint half 20a are fit tightly yet comfortably over the amputee's limb 15 in the overlapping arrangement as shown and described above. The splinting means 20 is secured to the limb 15 by securing means 30, which in the preferred embodiment are fabric hook-and-loop fastener cinch straps sold under the trade name VELCRO™ by Velcro USA Inc., 406 Brown Avenue, Manchester, N.H. 03103. The securing means 30 are affixed to the splinting means 20 by attachment means 35, preferably speed rivets such as those manufactured by Avdel USA LLC, 614 NC Highway 200 South, Stanfield, N.C. 28163 under the product line BRIV™ SPEED FASTENING™ System. The optional use of adhesive padding, preferably a moleskin blister cover (not shown), may be used to cover the rivets 35 protruding on the inside of the splinting means 20 in order to prevent chaffing and improve the comfort of fit. The rivets 35 can be placed through the manufactured perforations of the preferred POLYFORM™ material or an awl, rotary punch or similar device may be used to prepare the securing means 30 for attachment to the splinting means 20.

Moving now to the arm 40 of the embodiment of the invention shown in FIG. 1, we see that it is comprised of a pair of elongate arms (43,47) pivotally joined at a hinge 50. A proximal static elongate arm 43 is fixedly secured to the splinting means 20 by attachment means 35 through arm apertures 53 along the midline of the humerus of the limb 15 so that the hinge 50 extends preferably 1 to 2 inches beyond the distal end of the limb 15. Thus, the static arm 43 functions as the lower portion of the patient's upper arm. The hinge 50 functions as the elbow. The remaining distal mobile elongate arm 47 functions as the forearm. In the preferred embodiment of the invention, the arm 40 is a well known phoenix elbow hinge, such as that sold by RehabMart, LLC, 150 Sagewood Drive, Winterville, Ga. 30683 as model no. NC-15453.

Continuing the discussion of the arm 40 of the preferred embodiment of the invention, the mobile arm 47 also has manufactured apertures (60, 62) near the distal end of the mobile arm 47 and along its midline. A connecting means 72, preferably 50 lb test monofilament, is attached at the distal aperture 62 of the mobile arm 47 and threaded through an eyelet 70 which is fixedly attached to the splinting means 15. The connecting means 72 may be attached to an optional anchoring means 73, such as a block of foam for tucking into the amputee's waistline. Alternately, the connecting means 72 may be secured directly to the amputee's clothing (not shown) as, by way of example, to a belt loop.

Redirecting attention to the hinge 50 of the arm 40 shown in FIG. 1, we see that the hinge 50 is characterized by both an adjustable extension 80 and adjustable flexion 83 motion stop. The extension motion stop 80 is placed at an area on the hinge 50 distal to the static arm 43 (behind the mobile arm 47), in such a way as to prevent hyper-extension of the mobile arm 47. The flexion motion stop 83 is placed behind and near the static arm 43 to prevent hyper-flexion during use. In order to assist with extension of the mobile arm 47, an elastic element 90 securely connects the mobile arm 47 to the hinge 50. In FIG. 1, the elastic element 90 is a rubber band wrapped around the mobile arm 47 at a first end 93 and hooked around the extension motion stop 80 at a second end 95.

A cuff 100 for receiving a sliding element 105 is fixedly attached to the distal end of the mobile arm 47 of the preferred embodiment of the invention as shown in FIG. 1. The sliding element 105 is manufactured to receive a utensil attachment 110 by allowing the operator (not shown) to insert an attachment means 112, preferably a screw and wing nut, through an elongate slot 115 which allows for the variable placement of the attachment 110 along the longitudinal axis of the sliding element 105. Using the combination of the cuff 100, the sliding element 105, the elongate slot 115, and the attachment means 112, the configuration of the prosthetic advice is infinitely adjustable to meet a variety of body types, functions and circumstances, thereby augmenting the overall utility of the device.

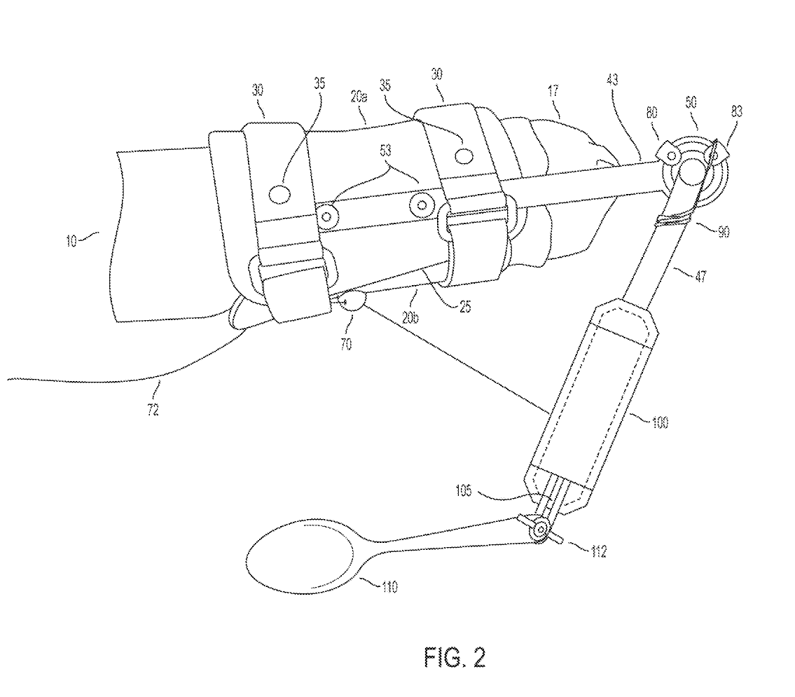

Turning now to FIG. 2, we see an overhead, plan view of an embodiment of the invention 10 in use with a spoon utensil attachment 110. By moving the patient's residual arm 15 through shoulder flexion and/or abduction, the connection means 72 translates the force of the shoulder flexion and/or abduction to its attachment point on the mobile arm 47, causing the mobile arm 47 to pivot towards the operator (not shown) around the hinge 50, thereby positioning the utensil attachment 110 into the desired position for functional activities. The length of the connection means 72 may be adjusted to accommodate for an increase or decrease in the range of motion of the residual limb 15. The elastic element 90 provides the tension necessary to facilitate extension of the mobile arm 47 after the force on the connection means 72 is reduced or discontinued entirely. The motion stops (80, 83) can be secured in various positions to lock the hinge 50 in a particular position to facilitate the use of the device for a particular activity such as, by way of example, writing, typing, and changing television stations. The use of spoon as a utensil device 110 is only one example of a utensil attachment that may be developed for use with this invention.

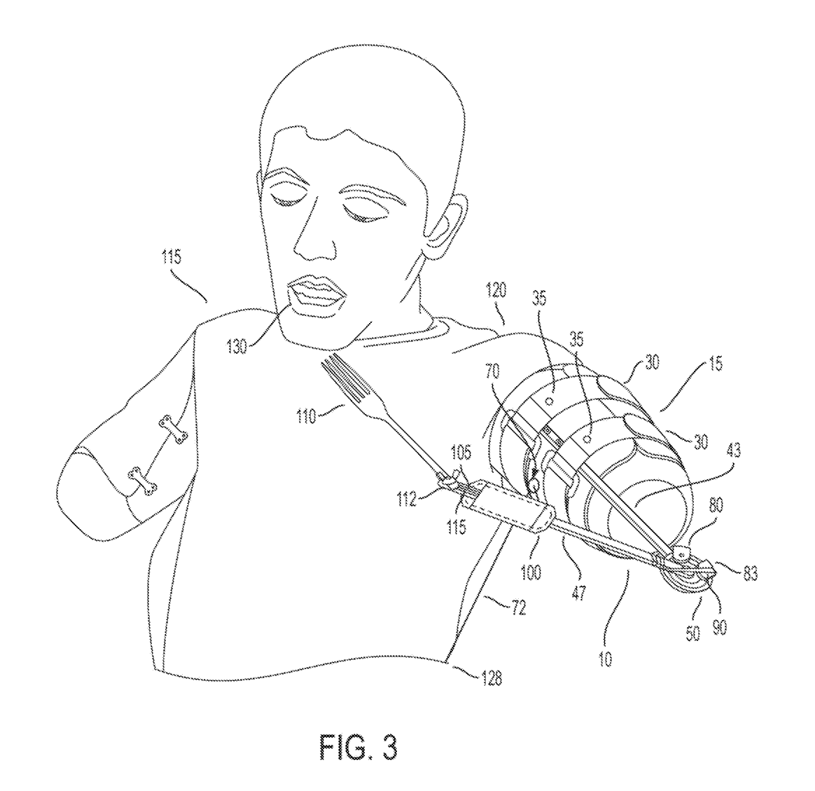

On FIG. 3 we see an embodiment of the invention mounted on the left residual limb 15 of an amputee 115. In this example, the embodiment of the invention 10 has been fitted with a fork utensil attachment 110 by attachment means 112 to the sliding element 105. By adjusting the angle along the plane of rotation created by the intersection of the sliding element 105 and the utensil attachment 110, the utensil attachment 110 may be adjusted as necessary to serve its intended function, in this instance, for eating. Further adjustments may be made by adjusting the attachment means 112 along the axis of the sliding element 105 using the elongate slot 115. Yet further adjustment may be made by adjusting the sliding element 105 within the cuff 100. The amputee 115 is using the movement of his shoulder 120 to create force along the connecting means 72 attached to his waist 128 by various means. The force of the shoulder 120 flexion and/or abduction is translated along the connecting means 72, through the eyelet 70 to the mobile arm 47 causing is to flex along the hinge 50 towards the amputee 115, thereby bringing the utensil attachment 110 closer to the amputee's mouth 130. The degree of flexion and extension of the mobile arm 47 can be limited by the strategic placement of the motion stops (80, 83). The elastic element 90 assists in the re-extension of the mobile arm 47 to reset the prosthetic device 10 to receive more food (not shown) using the fork utensil attachment 110.

While a specific embodiment of the invention has been shown and described in detail to illustrate the application of the principles of the invention, it will be understood that the invention may be embodied otherwise without departing from such principles.

### REFERENCES

The contents of each of which, and the contents of every other publication, including patent publications such as PCT International Patent Publications, being incorporated herein by this reference.
Lake C, D. R. (2006). Progressive upper limb prosthetics. Phys Med Rehabil Clin N Am, 17 (1), 49-72.

What is claimed is:

1. A prosthetic device comprising:
splinting means;
an elongate static arm fixedly attached to said splinting means;
an elongate mobile arm with a first end and second end, said mobile arm being flexibly attached at said first end to said static arm;
an elastic element for allowing re-extension of said mobile arm and attached to said mobile arm and an anchor point;
means for translating gross body movements to the mobile arm; and
an attachment being attached to the second end of the mobile arm.

2. The device of claim 1, wherein the splinting means comprise a clam-shell splint.

3. The device of claim 1, wherein the splitting means comprise thermoplastic.

4. The device of claim 1, wherein the elongate static arm is attached to said splinting means by hook-and-loop fastener cinch straps.

5. The device of claim 1, wherein the first end of said mobile arm is attached to the static arm by way of a hinge.

6. The device of claim 5, wherein the hinge is a phoenix hinge.

7. The device of claim 6, wherein the phoenix hinge further comprises an extension motion stop and a flexion motion stop.

8. The device of claim 1, wherein the means for translating gross body movements comprises an element with a first end and a second end, said first end of the element being attached to an anchor point and said second end of the element being attached to the mobile arm.

9. The device of claim 8, wherein the element is directed through a channel.

10. The device of claim 1, wherein the attachment is removably and adjustably attached to the second end of the mobile arm.

11. The device of claim 10, wherein the attachment is removably and adjustably attached to the second end of the mobile arm by means of a screw threaded through the attachment and a slotted elongate strip attached to the mobile arm.

12. A prosthetic device comprising:
splinting means secured to a residual limb by securing means;
an elongate static arm fixedly attached to the splinting means;
a hinge attached to the static arm and an elongate mobile arm, said hinge further comprising an extension motion stop and a flexion motion stop and an elastic element connecting the hinge to the mobile arm;
an eyelet attached to the splinting means;
connecting means with a first end and second end, said first end being attached to an anchor, said connecting means being threaded through the eyelet and said second end being attached to the elongate mobile arm;
a cuff for receiving a sliding element fixedly attached to the distal end of the mobile arm, the sliding element further characterized with an elongate slot and interoperable with the cuff; and,
a utensil attachment removably attached to said sliding element through the elongate slot by attachment means.

13. A method of providing self-sufficiency to an amputee comprising the steps of:
assessing the amputee as a qualifying candidate for prostheses;
preparing the residual limb to receive a splint;
splinting the residual limb;
attaching a static arm to said splint;
attaching a mobile arm to said static arm by way of a hinge;
attaching a flexible element to said mobile arm and an anchor point;
attaching means for translating the gross body movements of the amputee to the mobile arm and an anchor point; and,
attaching a utensil device to the mobile arm.

14. The method of claim 13 further comprising the step adjusting an extension motion stop and a flexion motion stop on said hinge to accommodate a particular activity to be performed by the amputee.

15. The method of claim 13 further comprising the step of training the amputee in the use of the prostheses.
